(19) United States
(12) Patent Application Publication
(10) Pub. No.: US 2015/0230970 A1
(43) Pub. Date: Aug. 20, 2015

(54) Dental Sleep Apnea Device

(71) Applicant: Albert H. Kaner, Los Angeles, CA (US)

(72) Inventor: Albert H. Kaner, Los Angeles, CA (US)

(21) Appl. No.: 14/181,432

(22) Filed: Feb. 14, 2014

(51) Int. Cl. A61F 5/56

(52) U.S. Cl. CPC A61F 5/566; A61F 2005/563

(57) ABSTRACT

The dental sleep apnea device and method utilizes a bite block (34) and a clear plastic tongue restrictor (38, 138) which is adjustable on the bite block by a coupling between a connecting rod with a spring-biased button assembly (46) that is capable of accommodating differing users' tongues (30). Sleep apnea is avoided by preventing the tongue from falling back during sleep and blocking the airway, and avoiding the creation of a diminished air supply, which can result a cessation of breathing while, at the same time, permitting the tongue to slide in an otherwise normal fashion. The tongue is thus not trapped in an uncomfortable manner and, therefore, during sleep, the patient can swallow because the tongue is allowed to move freely. When the tongue is so freely restricted, the patient will thus be able to rest as required and desired. The tongue restrictor coupling, when provided with swiveling and linear motion, enables the tongue restrictor to have a standard and fixed configuration which is couplable with any specifically formed bite block for multiple patients having differently sized mandibles (lower jaws).

### CROSS-REFERENCE TO RELATED APPLICATION

This application claims the benefit of U.S. Provisional Application No. 61/764,535, filed 14 Feb. 2013.

### REFERENCE REGARDING FEDERAL SPONSORSHIP

Not Applicable

### REFERENCE TO MICROFICHE APPENDIX

Not Applicable

### BACKGROUND OF THE INVENTION

1. Field of the Invention

The present invention relates to an apparatus and a method relating to sleep apnea and, in particular, to improvements in such apparatus and method in which a person's tongue is prevented from falling back into the airway during sleep, by restricting the tongue's ability to do so, but not to impede otherwise movement of the tongue, that is, to permit the tongue to move freely as normal, with the exception of its falling backward, so as to enable as little disturbance to the person's sleep.

2. Description of Related Art and Other Considerations

Known apparati and methods physically hold the tongue captive, e.g., by using a vacuum or a tongue tip holder device that holds the tip of the tongue, rendering the tongue immobile. Adjustment of the position of lower jaw, or locking the tongue into position in any fashion is unwise, e.g., for the following reason. Tampering with the TMJ (temporomandibular joint), moving the lower jaw or opening the bite or locking the tongue into position by the suction is, as described in some prior art devices, at least uncomfortable and possibly harmful to the user. As others have attempted to lock the tongue in place by holding the tip of the tongue, the prior existing art creates unnecessary problems. Thus, when the tongue is prevented from moving normally during sleep, it interferes with the sleep that all require. By changing the position of the jaw, either opening the bite or moving the lower jaw forward, the anatomic orientation of the mouth during sleep is unnecessarily changed.

Some further concerns may occur in prior art CPAP and BIPAP apparatus, that employ masks which are coupled to air-pumping machines and in which, by providing air for the airway, several problems can arise from their use, such as the formation of cancer or mold due to the difficulty of their being able to be cleaned. Nasal bubbles may form with an airway device, and it may be difficult to balance proper pressure for keeping bubbles in position to provide an adequate airway to the patient, and the equipment used is not well designed to allow proper cleaning. The tongue may still be blocked if not physically held and prevented from occluding the airway. In addition, such prior art apparatus is considered by some to be unattractive, perhaps even frightening, and to be at least an impediment to conversation.

### SUMMARY OF THE INVENTION

The present invention avoids and overcomes the aforementioned and other problems. Two appliances are used, a bite block and a clear plastic restricting plate. The bite block is constructed of a plastic tray, which fits over the lower teeth. It is preferably made usually from methyl-methacrylate, which is a hard plastic and which is used in denture fabrication. The biting surface of this bite block is made of a softer plastic, which occludes with the upper teeth. This softer plastic is used to prevent bruxism, that is, a clenching and grinding of the teeth during sleep, which can result in damage to the upper, opposing teeth.

The clear plastic restrictor plate is constructed to cover the tongue, without physically holding the tongue captive, (e.g., by using a vacuum, as some prior devices have attempted, or by a tongue tip holder device that holds the tip of the tongue which renders the tongue immobile). The restrictor plate, which is also made of a clear hard plastic as the bite block material (but, if desired, colored differently from the bite block), only blocks the tongue to prevent it by falling backwards. During sleep, the patient must be allowed to swallow, allowing the tongue to move freely (e.g., from side to side). When the tongue is so restricted, the patient will thus be able to rest as required and desired. This applies to altering the position of the lower jaw, by moving it forward, or opening the bite. These and other problems are successfully addressed and overcome by the present invention.

The restrictor plate has, preferably for example, four rods. Two rods are attached on the inner left (lingual) side of the bite blocks on the anterior between the first and second bicuspids. Two more rods are attached on the inner right side (lingual) of the bite block between the first and second lower (mandibular) bicuspids. The bottoms of the rods are fitted with spring buttons. When the rods are positioned in the corresponding cylinders in the bite blocks, they can be attached to the first, second, third, or fourth opening on the cylinder and raised or lowered, as applicable, to accommodate differently sized tongues. When the spring button, the locking device, is positioned on the bottom (4th) opening, the restrictor plate is closest to the top of the tongue. As the restrictor plate moves up, by the attachment using the spring button to snap into the third, second, or first opening, this alternative attaching structure allows proper spacing to accommodate smaller and larger tongues.

Several advantages are derived from this arrangement. The presently described apparatus can accommodate differently sized tongues while prior art devices do not. This design allows simple adjustments of the tongue restrictor to be made and a visual check by the dentist to ascertain the most comfortable position of the restrictor. A visual check is enabled by means of the clear plastic restrictor so that the dentist can visually ascertain the most comfortable position for the restrictor. The tongue is not held hostage, which allows the patient to sleep comfortably and without being disturbed. Impediments to speech or conversation, as a problem with at least some prior art devices, is minimized, if not entirely avoided. The imputed unattractiveness of some prior art devices is avoided.

Other aims and advantages, as well as a more complete understanding of the present invention, will appear from the following explanation of exemplary embodiments and the accompanying drawings thereof.

### BRIEF DESCRIPTION OF THE DRAWINGS

FIG. 1 is an illustration of a user's oral cavity and oropharynx showing, in phantom, the receiving cylinders on the bite block as later depicted in the following figures, see, e.g., FIGS. 2A and 4;

FIGS. 2A and 2B are a combined view of major components of the present invention comprising (a) a bite block with receiving cylinders integrated therewith which bite block is form-fitted to encompass the mandibular (lower jaw) teeth and (b) a clear plastic tongue restrictor with rods having spring-loaded or biasing buttons integrated therewith for selective engagement with the bite block receiving cylinders, with each bite block receiving cylinder having openings to selectively engage a tongue restrictor rod spring-loaded, biasing button;

FIGS. 2a and 2b are a combined view of mayor components of the present invention comprising (a) a bite block with receiving cylinders integrated therewith which bite block is form-fitted to encompass the mandibular (lower jaw) teeth and (b) a clear plastic tongue restrictor with rods having spring-loaded or biasing buttons (figuratively shown as springs) integrated therewith for selective engagement with the bite block receiving cylinders, with each bite block receiving cylinder having openings to selectively engage a tongue restrictor rod spring-loaded, biasing button;

FIG. 3 is a top view of the tongue restrictor with its rods inserted through its spring-loaded or biasing buttons for enabling an engagement with the bite block receiving cylinders;

FIG. 4 is a top view in partial cross-section of the bite block with the tongue restrictor engaged therewith with its integrated rods with its spring-loaded, biasing buttons engaged with the bite block receiving cylinders;

FIGS. 5, 6A, 6B, 7A and 7B are respective views of an individual bite block receiving cylinder, a tongue restrictor rod with its biasing spring-loaded buttons, and the coupling between the rod and the cylinder, in which FIGS. 6A and 6B are alternate views of the same connecting rod and FIGS. 7A and 7B are alternate views of the same combined connecting rod with spring button inserted within the receiving cylinder;

FIG. 8 is a perspective illustration depicting framework connectors enabling the engagement of the tongue restrictor with the bite block through their respective spring-loaded (biased) buttons and receiving cylinders;

FIG. 9 is a view of another embodiment of the present invention illustrating the use of a specially made bite block which is fitted to an individual user's lower teeth and a universal tongue restrictor that is usable with any configured bite block, as molded to differently sized and shaped jaws, though the use of pivotally and linearly movable connecting rod mechanisms;

FIG. 10 is a view similar to that of FIG. 9 depicting per se the universal tongue restrictor that is usable with any configured bite block though the use of pivotally and linearly movable connecting rod mechanisms;

FIG. 11 is a cross-sectional view of a portion of the universal tongue restrictor taken along line 11-11 of FIG. 9; and

FIG. 12 is a second embodiment of the present invention illustrating an alternate engagement of the bite block to the lower teeth.

### DETAILED DESCRIPTION OF THE PREFERRED EMBODIMENTS

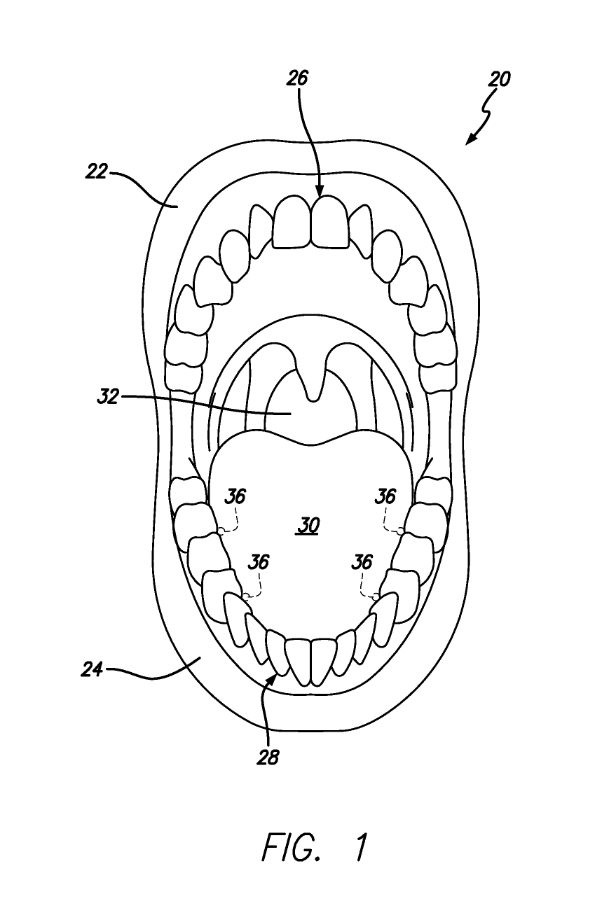

Accordingly, as depicted in FIG. 1, a user's oral cavity and oropharynx 20 is shown as including upper and lower lips 22 and 24, upper (maxillary) and lower (mandibular) teeth 26 and 28, a tongue 30, and an airway or air passage 32, among other parts.

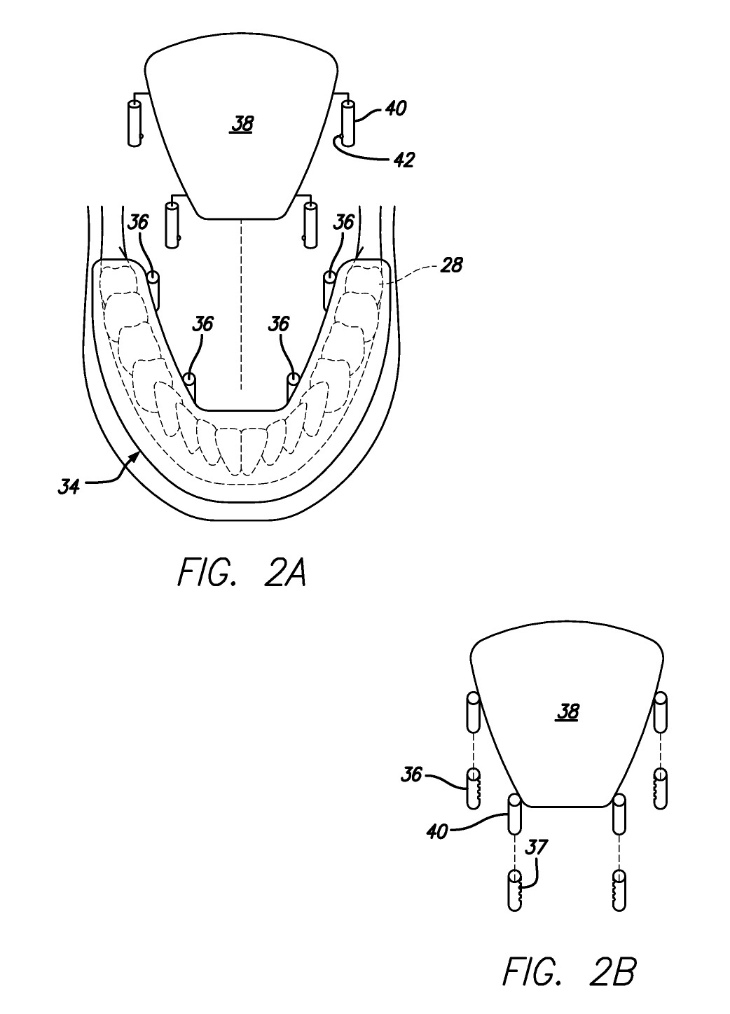

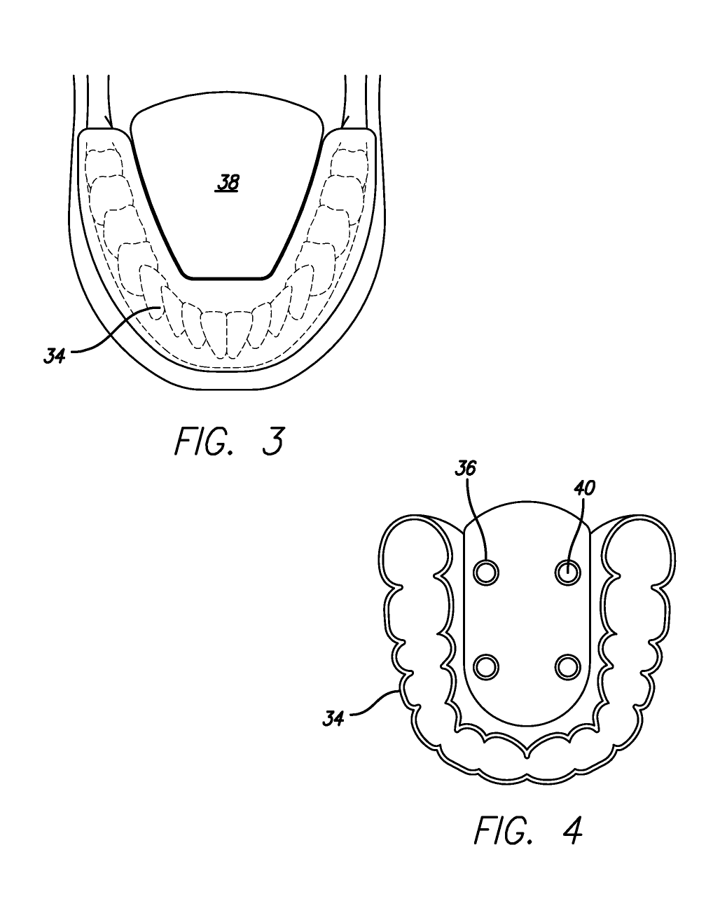

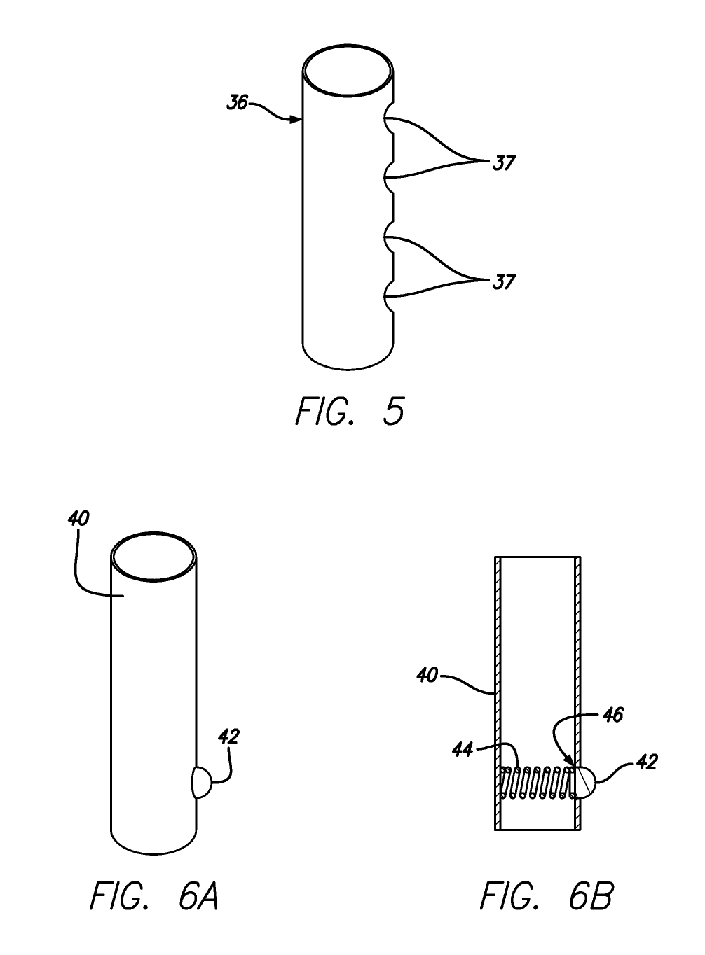

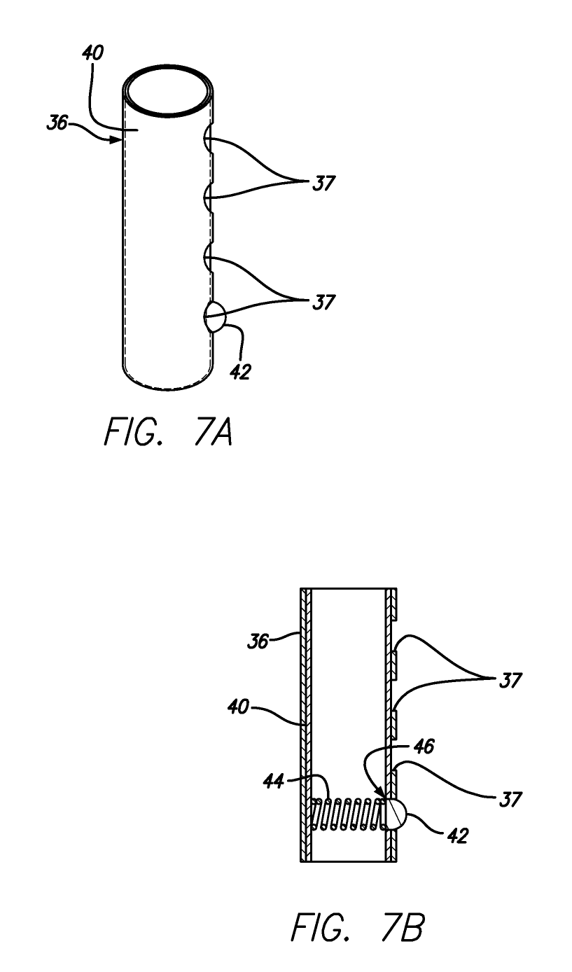

Referring to FIGS. 2A and 2B, the apparatus and a method relating to sleep apnea, as embodied in the present invention, is embodied as a bite block 34 which is configured and molded to encompass lower teeth 28. Attached to bite block 34 are a plurality of receiving cylinders 36, preferably four in number, appropriately positioned within the interior of the bite block. Each receiving cylinder includes a plurality of holes 37 extending along its length (see, e.g., FIG. 5). A tongue restrictor 38 is positioned above the bite block and has a plurality of connecting rods 40, preferably four in number, appropriately placed about the exterior of the tongue restrictor and aligned with receiving cylinders 36. Tongue 30 is disposed to reside under tongue restrictor 38 and generally between bite block 34. Connecting rods 40 see also FIGS. 6A and 6B) include a button 42 backed up by a spring or other biasing element 44, in combination forming a spring button assembly (or spring-biased button assembly) 46. When each connecting rod is inserted into its aligned receiving cylinder, its button 42 snaps into one or more receiving cylinder holes 37 as urged by its spring 44 which is functions as an adjustable arrangement to provide spacing to accommodate the different thicknesses of all users' tongues. As best illustrated in FIGS. 1, 2A and 4, two rods 40 are attached on the inner left (lingual) side of the bite blocks on the anterior between the first and second bicuspids. Two more rods 40 are attached on the inner right side (lingual) of the bite block between the first and second lower (mandibular) molars.

In the preferred embodiment, the surface of the bike block 34 is positioned directly on top of bottom teeth 28 and can articulate with respect to upper teeth 26. The bite block is constructed as a plastic tray, which fits over the lower teeth. It is made, usually from methyl-methacrylate, a hard plastic, which is used in denture fabrication. The biting surface of this bite block is made of a softer plastic, which occludes with the upper teeth. This softer plastic is used to prevent bruxism, i.e., a clenching and grinding of the teeth during sleep, which can result in damage to the upper, opposing teeth.

Tongue restrictor 38 can thus be so shaped and the sides can thus be so positioned at the bottom of the sulcus, that is, the position where the bottom of the tongue joins the bottom of the gums. As a consequence, the containment of the tongue is not accomplished in a harsh manner as some concepts of the prior art, such as by holding the tongue hostage by means of vacuum device which may inflict pain on the patient or secondly, by holding the tip of the tongue with a mechanical device to actually prevent its free motion during sleep. In the present invention, the user's tongue is permitted to move as freely as normally possible, with the exception of its falling backwards in the patient's air passageway 32, which otherwise would result in sleep apnea and interfere with the person's sleep. In short, the intent is that tongue 30 should not be held in any manner physically, which can cause a disruption in sleep. The primary reason for restricting the tongue is simply to prevent the tongue, during sleep, from falling back and occluding the airway, which would cause sleep apnea. If desired or needed, supplemental oxygenation methods and apparatus can be employed.

Further, tongue restrictor 38 preferably incorporates a clear plastic piece to enable the sleep apnea practitioner to observe the position of the tongue or plate. By questioning the patent, the most comfortable position for that patient can be easily determined. In addition, the tongue restrictor may be curved to conform to the user's tongue.

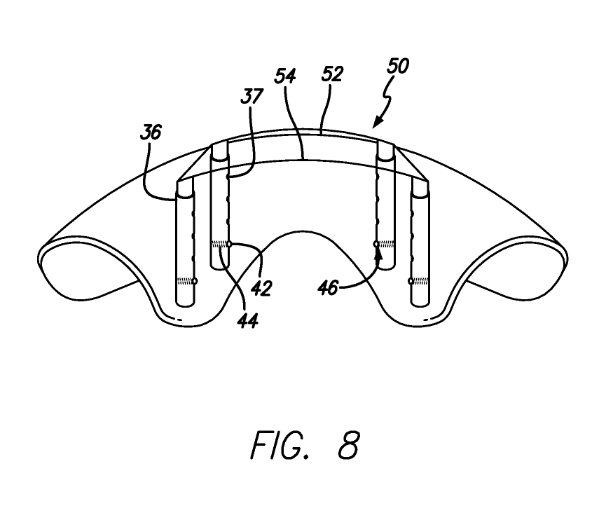

As depicted in FIG. 8, a frame work 50 includes anterior and posterior transverse connectors 52 and 54 upon which tongue restrictor 38 is secured. Spring button assembly 46 depends from frame work 50 for coupling the tongue restrictor to the bite plate. As such, the tongue restrictor must be configured to each bite plate, in accordance with each user's or patient's mouth.

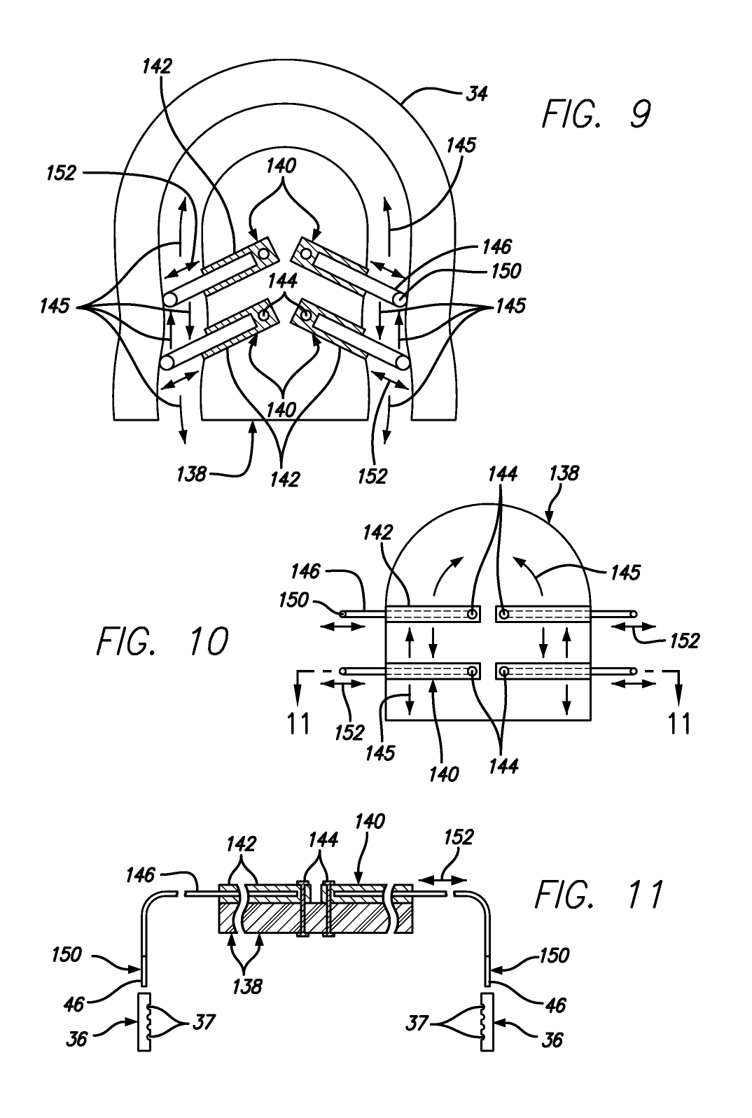

To avoid the need to utilize a specially and individually configured tongue restrictor as described and illustrated in FIG. 8, reference is made to the construction shown in FIGS. 9-11 which construction depicts a tongue restrictor 138 to have a configuration which is universal and standardized and, thereby, which is adaptable to any specifically designed bite block. Here, the bite block, such as is identified by indicium 34, is specifically molded and configured for a specific user. In place of spring-loaded or biasing implementation shown as connecting rods 40 and frame work 50, as previously described and shown, FIGS. 9-11 depict a biasing, spring-loaded implementation 140, numbering four assemblies, each comprising a tubular member 142 which is pivotally mounted on tongue restrictor 138 by a pivot 144. Thus, each tubular member 142 can pivot with respect to the tongue restrictor as depicted by arrow-headed lines 145. Each spring-loaded biased implementation is completed by a rod 146 terminating in a 90° terminus 150 in which a spring button assembly 46 is housed. Each rod 146 in housed within its tubular member 142 for linear movement therein, as denoted by double-headed straight arrow lines 152. Therefore, regardless of the specific construction of bite block 34 and the placement of its receiving cylinders 36, each tubular member 142 can be pivoted along with its rod 146 and terminus 150 which thereby can be manipulated and positioned to engage receiving cylinders 36.

To avoid the need to utilize a specially configured tongue restrictor as described and illustrated in FIGS. 8a and 8b, reference is made to the construction shown in FIGS. 9-11 which depicts a tongue restrictor 138 to have a configuration which is universal and standardized and, thereby, which is adaptable to any specifically designed bite block. Here, the bite block, such as is identified by indicium 34, is specifically molded and configured for a specific user. In place of spring-loaded or biasing implementation shown as connecting rods 40 and frame work 50, as previously described and shown, FIGS. 9-11 depict a biasing, spring-loaded implementation 140, numbering four assemblies, each comprises a tubular member 142 which is pivotally mounted on tongue restrictor 138 by a pivot 144. Thus, each tubular member 142 can pivot with respect to the tongue restrictor as depicted by curved arrow-headed lines 145. Each spring-loaded biased implementation is completed by a rod 146 terminating in a 90° terminus 150 in what a spring button assembly 46 is housed. Rod 146 in housed within its tubular member 142 for linear movement therein, as denoted by double-headed straight arrow lines 152. Therefore, regardless of the specific construction of bite block 34 and the placement of its receiving cylinders 36, each tubular member 142 can be pivoted and its rod 148 with terminus 150 can be manipulated to engage receiving cylinders 36.

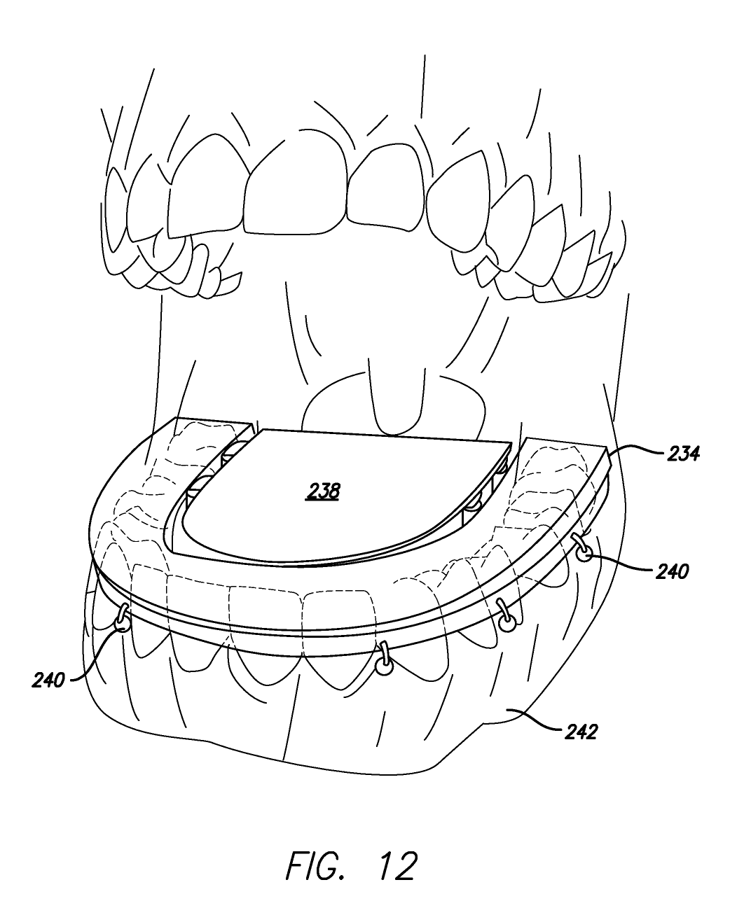

Referring now to FIG. 12, in place of the previously described embodiment, the coupling is between a tongue restrictor 238 and a bite block 234 which is clasped by clasps 240 to the lower jaw (242).

While the preferred connection between the bite block and the tongue restrictor comprises a rod with a spring button assembly fittable within one of a series of openings in the bite block receiving cylinders, it is to be understood that other biasing mechanisms may be employed.

Further, it is believed that stability of the tongue restrictor is best achieved through the described positioning of the four receiving cylinders (a) in the bite block adjacent the inner left (lingual) side of the bite blocks on the anterior between the first and second bicuspids and (b) the inner right side (lingual) of the bite block between the first and second lower (mandibular) molars. However, should it be found that such positioning of the four receiving cylinders be achieved otherwise, e.g., against other teeth, such finding is encompassed within the spirit and scope of the invention.

Although the invention has been described with respect to particular embodiments thereof, it should be realized that various changes and modifications may be made therein without departing from the spirit and scope of the invention.

What is claimed is:

1. A dental sleep apnea device adaptable to engaging a user's teeth for restricting the user's tongue from movement into user's air passage comprising:
a bite block adaptable for engagement with the user's teeth; and
a tongue restrictor engageable with the bite block holding the user's tongue from moving into the user's air passage.

2. A dental sleep apnea device according to claim 1 wherein said bite block and said tongue restrictor include engagement mechanisms providing spacing therebetween to accommodate the thickness of the user's tongue.

3. A dental sleep apnea device according to claim 2 wherein said engagement mechanisms comprise cylindrical implementation secured to one of said bite block and said tongue restrictor and biasing implementation secured to the other of said bite block and said tongue restrictor, whereby said cylindrical and biasing implementations are engageable to space said tongue restrictor from said bite block and, thereby, to accommodate the thickness of the user's tongue.

4. A dental sleep apnea device according to claim 3 wherein said cylindrical implementation comprises a plurality of cylinders having holes spaced along their lengths, and said biasing implementation comprises a plurality of rods with biasing buttons engaged with selective ones of said bite block receiving cylinders.

5. A dental sleep apnea device according to claim 4 wherein said bite block is individually configured to an individual user and wherein, for each of said rods, said biasing implementation further comprises a tubular enclosure pivotally secured to said tongue restrictor and disposed to enclose its rod to enable said rod to linearly slide within said tubular enclosure and to be aligned with each of said bite block cylinders, whereby said tongue restrictor results in being universally adaptable to any said bite block however individually configured.

6. A dental sleep apnea device according to claim 1 wherein said bite block is adapted for engagement with the user's lower jaw.

7. A dental sleep apnea device according to claim 2 wherein said bite block is adapted for engagement with the user's lower jaw.

8. A dental sleep apnea device according to claim 3 wherein said bite block is adapted for engagement with the user's lower jaw.

9. A dental sleep apnea device according to claim 4 wherein said bite block is adapted for engagement with the user's lower jaw.

10. A dental sleep apnea device according to claim 5 wherein said bite block is adapted for engagement with the user's lower jaw.

11. A dental sleep apnea device according to claim 2 wherein said bite block comprises a rim which is joined by clasps to the user's lower jaw.

12. A dental sleep apnea device according to claim 11 wherein said bite block is adapted for engagement with the user's lower jaw.

13. A dental sleep apnea method for restricting the user's tongue from movement into user's air pipe comprising the steps of:
adopting a bite block to engage the user's lower jaw; and
engaging a tongue restrictor with the bite block to hold the user's tongue from moving into the user's air passage.

14. A method according to claim 13 further including the steps of providing a variable engagement between the bite block and the tongue restrictor and a spacing therebetween to accommodate the thickness of different user's tongues.

15. A method according to claim 14 comprising the step of providing the bite block with a coupler mechanism and in which said variable engagement steps further comprise the steps of providing a swiveling and linearly movable implementation coupled to the tongue restrictor for engaging the bite block coupler mechanism with the swiveling and linearly movable implementation, thus enabling the tongue restrictor to mate with the bite block regardless of its configuration and its coupler mechanism, thus configuring the tongue restrictor as one having a fixed, universal shape.
